(19) United States
(12) Patent Application Publication
(10) Pub. No.: US 2025/0099037 A1
(43) Pub. Date: Mar. 27, 2025

(54) RING-SHAPED COMPUTER DEVICE

(71) Applicant: ONVY HealthTech Group GmbH, Grünwald (DE)

(72) Inventor: Julius RACHOR, München (DE)

(21) Appl. No.: 18/730,567

(22) PCT Filed: Jan. 19, 2023
(86) PCT No.: PCT/DE2023/100041

§ 371 Date: Jul. 19, 2024

(30) Foreign Application Priority Data
Jan. 21, 2022 (DE) 10 2022 101 446.0

(51) Int. Cl. A61B 5/00; G06F 1/16

(52) U.S. Cl. CPC A61B 5/6826; G06F 1/163; A61B 2560/0214; A61B 2562/063; A61B 2562/166

(57) ABSTRACT

A ring-shaped computer device for wearing on the human body, in particular on a finger, which computer device is configured to continuously collect vital parameters and biomarkers of a wearer and make them available to a connected device and includes a plurality of sensors for recording the vital parameters and biomarkers, a printed circuit board for receiving the sensors and a data interface, a processor, and at least two battery modules for supplying power to the printed circuit board and the processor, which are connected together to form an overall battery.

### FIELD OF THE INVENTION

The invention relates to a ring-shaped computer device for wearing on the human body, in particular on a finger, which computer device is configured to continuously collect vital parameters and/or biomarkers of a wearer and make them available to a connected device.

### BACKGROUND

In the current state of the art, wearable computer devices are already known which are worn on the finger and collect vital parameters and/or biomarkers of a user. Vital parameters can be physical parameters such as blood pressure. Biomarkers can be chemical substances that can indicate physiological conditions.

To ensure that the wearable computer device remains functional over a longer period of time without an external power supply, such devices have a battery integrated in the device. This battery is curved to fit the shape of the device. This is considered to be particularly space-saving.

However, the weight of the battery makes up a significant proportion of the total weight of the device. The battery can therefore cause the centre of gravity of the device to be positioned outside the axis of symmetry of the device body. This in turn means that the device has a greater tendency to slip when worn and may therefore interfere with data acquisition by the sensors.

As currently available batteries can only be bent to a limited extent without restricting their functionality, the dimensions of the device and the dimensions of the battery must be coordinated in such a way that the battery only has a relatively small radius of curvature when the device is in the assembled state. Consequently, in the case of a curved basic shape of the device, only part of the interior space can be filled with the battery, which in turn leads to a less than optimal utilisation of space.

The problem addressed by the invention is that of at least minimising these disadvantages.

### BRIEF DESCRIPTION OF THE INVENTION

This problem is solved by the invention specified in the independent claims. Advantageous embodiments can be found in the dependent claims.

According to the invention, a ring-shaped computer device of the type mentioned at the outset is created, comprising a plurality of sensors for recording the vital parameters and/or biomarkers; a printed circuit board for receiving the sensors and a data interface; a processor; and at least two battery modules for supplying power to the printed circuit board and the processor, which are connected together to form an overall battery.

Traditionally, curved batteries are used, but their radius of curvature is limited. However, a computer device in the form of a finger ring, which is intended to enclose a battery, is strongly curved, so that the curvature of the ring is greater than the curvature of the battery. If the curvature of the battery is to be adapted to the curvature of the ring, this is only possible at the expense of battery performance. If the curvature of the battery is less than that of the ring, this results in poor utilisation of installation space at the end regions of the battery.

Therefore, instead of one curved battery module, at least two battery modules are installed, wherein the battery modules can have correspondingly smaller dimensions. This means that the curvature of the battery can be dispensed with if several smaller, non-curved batteries are installed instead. This also allows more powerful batteries to be installed. Interconnecting several smaller batteries or battery modules to form an overall battery results in better utilisation of installation space with increased performance of the overall battery. If several battery modules are installed, a more balanced mass distribution is also possible, which increases wearing comfort and the measuring accuracy of the sensors.

According to the invention, a distributed, segmented battery is therefore used, the segments of which form battery modules and which can be arranged distributed in an interior space of the computer device. This can influence the weight distribution within the computer device. In addition, a higher packing density in the interior space of the device can be achieved by using different sub-batteries, in particular if the device has a radially symmetrical main body. The invention thus creates a battery for the computer device which is particularly space-saving and at the same time leads to increased wearing comfort thanks to optimum weight distribution.

In one embodiment of the invention, the at least two battery modules are arranged in an interior of the computer device and are equally distributed over a circumference of the ring-shaped computer device, so that a centre of mass of the computer device is located in an imaginary circle centre, in particular on an axis of symmetry of the computer device.

The batteries are the heaviest components in the computer device and therefore have the greatest influence on any possible twisting of the computer device on the wearer's finger as a result of uneven mass distribution. Therefore, an even distribution of the battery mass around the circumference is the most effective way to prevent twisting and increase wearing comfort. In addition, the sensors of the computer device must be held in their predetermined positions to effectively take measurements. This is another reason why twisting of the computer device is undesirable and can be prevented by the embodiments according to the invention. However, the batteries can also be arranged in a radial direction.

In one embodiment of the invention, a weight distribution of the battery modules and all other installed components leads to an overall centre of gravity of the computer device, which is located in the immediate vicinity of at least one axis of symmetry of a device body, wherein the ring-shaped computer device forms a tube portion, and wherein the axis of symmetry is a longitudinal axis of the tube portion. In this embodiment, all components inside the computer device are arranged such that the centre of gravity of the entire computer device lies on the axis of symmetry. In other words, in order to further prevent unintentional twisting of the computer device, the masses of the other components are, additionally, also included, so that the overall centre of gravity of all components lies approximately on the longitudinal axis. As a result, no torques caused by the mass distribution of the components are generated when the computer device is worn on a finger. This further prevents the ring from twisting on the finger. This is particularly important during sport, for example when the finger gets wet due to sweating and the friction between the skin and the ring decreases.

In one embodiment of the invention, the overall battery comprises three battery modules and a sensor assembly, which are evenly distributed around a circumference.

If the battery modules and sensor assembly are positioned opposite each other on the circumference, mass-related torques in relation to the longitudinal axis cancel each other out. This leads to a balanced weight distribution. The more battery modules there are that are arranged evenly distributed around the circumference, the more balanced the mass distribution of the entire computer device will be. In addition, the packing density also increases with many small batteries and the utilisation of installation space is further improved.

In one embodiment of the invention, each of the battery modules has a rectangular cross-section and is cuboidal in shape. Rectangular batteries are the most readily available and the most powerful.

In one embodiment of the invention, each of the battery modules comprises a plurality of interconnected battery cells, wherein each of the battery cells is cuboidal and an extent in the circumferential direction is substantially less than a height in the radial direction.

The use of a large number of (small) rectangular, cuboidal batteries as on a chain increases the packing density and further improves the utilisation of installation space. The height of the battery in the radial direction should be significantly greater than the base side. This results in narrow battery cells in the circumferential direction, i.e. battery modules that are highly segmented and can therefore adapt well to the contour, i.e. the curvature of the ring-shaped computer device.

### BRIEF DESCRIPTION OF THE DRAWINGS

Exemplary embodiments of the invention are described in greater detail below with reference to the accompanying drawings, in which:

FIG. 1 shows a first exemplary embodiment of the invention;

FIGS. 2 and 3 show further exemplary embodiments of the invention; and

FIG. 4 shows a further exemplary embodiment of the invention.

### FIGURE DESCRIPTION

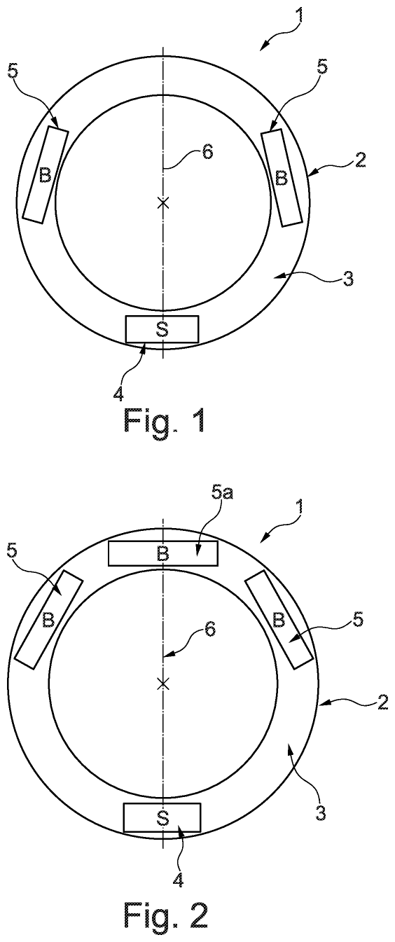

FIG. 1 shows a first exemplary embodiment of the invention. The wearable computer device 1 is ring-shaped and comprises a ring-shaped main body 2. The ring-shaped main body forms an interior space 3, an interior in which a sensor assembly 4 is arranged, which comprises further components such as a printed circuit board, a processor and a data interface (not shown). At least two battery modules 5 are arranged in the interior space and are interconnected to form an overall battery and supply the sensor assembly 4 with electrical energy. The battery modules 5 are arranged symmetrically on both sides in relation to a vertical axis of symmetry 6 of the computer device 1. This results in a position of the total centre of gravity on the vertical axis of symmetry 6. Thus, in the position shown, the torques twisting the computer device 1 due to uneven mass distribution are balanced.

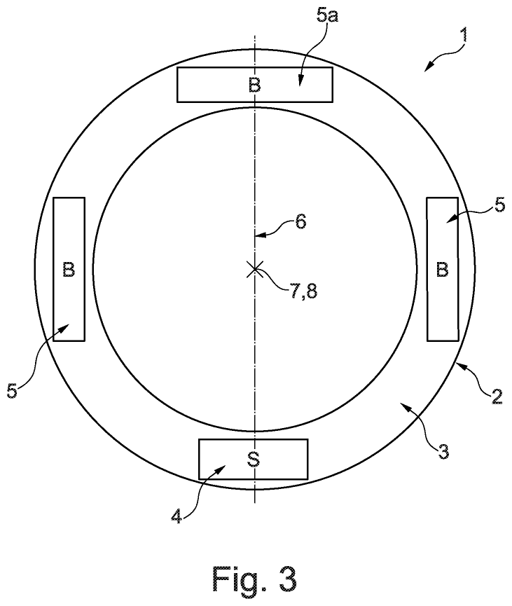

FIG. 2 shows a second exemplary embodiment of the invention. Three battery modules 5 are arranged symmetrically with respect to the vertical axis of symmetry 6, so that the centre of mass of the computer device 1 lies on the axis of symmetry 6. This also includes the mass of the sensor module 4. The upper battery module 5a is positioned opposite the sensor assembly 4 in order to balance its mass in relation to the centre of gravity.

FIG. 3 shows a third exemplary embodiment of the invention, wherein three battery modules 5, 5a are arranged inside the main body 2. The three battery modules 5, 5a and the sensor assembly 4 are equally distributed around the circumference and are arranged opposite each other in pairs. The centre of mass of the computer device 1 lies here in an imaginary circle centre 7, which is located on the vertical axis of symmetry 6. The computer device 1 forms a tube portion of which the longitudinal axis 8 runs through the circle centre 7 and intersects, therein, the vertical axis of symmetry 6. Each of the battery modules is cuboidal and has a rectangular cross-section. The dimensions of the cuboidal battery modules 5 are chosen so that as little installation space as possible is lost in the end regions. The narrower, i.e. the smaller the extent in the circumferential direction, the more efficiently the installation space in the interior 3 of the main body 2 can be utilised.

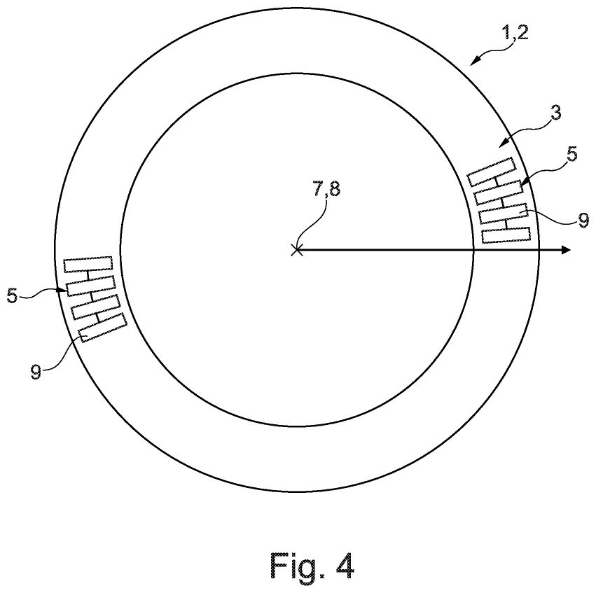

FIG. 4 shows a further embodiment of the invention, wherein two battery modules 5 lie opposite each other in the interior 3 of the main body 2. The battery modules 5 are formed from a plurality of battery cells 9, which are interconnected to form a battery module 5. The individual battery cells 9 are cuboidal and have a considerably greater extent in the radial direction (marked by an arrow in FIG. 4) than in the circumferential direction. Four of the narrow battery cells 9 are electrically and mechanically connected and form a chain-like structure. In order to utilise installation space, any number of battery cells 9 can be connected to form a battery module 5.

### LIST OF REFERENCE SIGNS

1 computer device
2 ring-shaped main body
3 interior space/interior of the main body
4 sensor assembly/sensors and other components
5 battery modules
5a upper battery module
6 vertical axis of symmetry
7 imaginary circle centre
8 longitudinal axis
9 battery cells

1. A ring-shaped computer device for wearing on the human body, in particular on a finger, which computer device is configured to continuously collect vital parameters and/or biomarkers of a wearer and make them available to a connected device and comprises:
a plurality of sensors for recording the vital parameters and/or biomarkers;
a printed circuit board for receiving the sensors and a data interface;
a processor; and
at least two battery modules for supplying power to the printed circuit board and the processor, which are connected together to form an overall battery.

2. The computer device according to claim 1, wherein the at least two battery modules are arranged in an interior of the computer device and are equally distributed over a circumference of the ring-shaped computer device, so that a centre of mass of the computer device is located in an imaginary circle centre, in particular on an axis of symmetry of the computer device.

3. The computer device according to claim 1, wherein a weight distribution of the battery modules and all other installed components leads to an overall centre of gravity of the computer device, which lies in the immediate vicinity of at least one axis of symmetry of the device body, wherein the ring-shaped computer device forms a tube portion, and wherein the axis of symmetry is a longitudinal axis of the tube portion.

4. The computer device according to claim 1, wherein the overall battery comprises three battery modules and a sensor assembly, which are arranged equally distributed over a circumference.

5. The computer device according to claim 1, wherein each of the battery modules has a rectangular cross-section and is cuboidal.

6. The computer device according to claim 1, wherein each of the battery modules comprises a plurality of interconnected battery cells, wherein each of the battery cells is cuboidal and an extent in the circumferential direction is substantially less than a height in the radial direction.
